(19) United States
(12) Patent Application Publication
(10) Pub. No.: US 2026/0174667 A1
(43) Pub. Date: Jun. 25, 2026

(54) COSMETIC COMPOSITION IN FORM OF A STABLE SUSPENSION

(71) Applicant: PIERRE FABRE DERMO-COSMÉTIQUE, LAVAUR (FR)

(72) Inventors: Laetitia LEMERCIER, CASTRES (FR); Céline ALIAGA, CASTRES (FR); Anne-Laure GAUDRY, CASTRES (FR)

(73) Assignee: PIERRE FABRE DERMO-COSMÉTIQUE, LAVAUR (FR)

(21) Appl. No.: 19/127,532

(22) PCT Filed: Dec. 19, 2023
(86) PCT No.: PCT/EP2023/086578

§ 371 Date: May 6, 2025

(30) Foreign Application Priority Data
Dec. 23, 2022 (FR) FR2214429

(51) Int. Cl. A61K 8/891; A61K 8/365; A61Q 19/10

(52) U.S. Cl. CPC A61K 8/891; A61K 8/365; A61Q 19/10

(57) ABSTRACT

The invention relates to a cosmetic composition for skin and/or hair care and/or hygiene. Said composition contains a surfactant mixture comprising at least one anionic surfactant selected from among alaninates, lauryl glucoside and at least one amphoteric surfactant, as well as water, a mixture of cellulose and cellulose gum and insoluble particles in stable suspension in the composition. In addition, the composition contains no amidopropyl betaines and no surfactant with a sulfate group.

### TECHNICAL FIELD OF THE INVENTION

The present invention relates to a novel cosmetic composition comprising particles in a stable suspended state and free from controversial substances, as well as the cosmetic use of such a composition for skin and/or hair washing and/or care. The invention also relates to a cosmetic treatment method for skin and/or hair hygiene and/or care using such a composition.

### PRIOR ART

A cosmetic composition intended for skin and/or hair washing and/or care must firstly be effective, in other words cleansing, foaming and where applicable detangling. Indeed, the main expectations of consumers are to have products which produce large amounts of foam and which, in the case of shampoos, have a hair detangling effect.

Furthermore, the addition of insoluble particles to cosmetic compositions is common. These particles can have different functions. Some have the ability to include active agents, in particular millicapsules. Others make it possible to opacify compositions, such as pearlescent bases, playing on the visually appealing aspect of cosmetic products. Finally, some particles have exfoliating properties, and are useful for scrubbing the skin, in particular the scalp. Cosmetic compositions including exfoliating particles make it possible to effectively remove dead skin, product residues, and excess sebum from the skin surface. They are ideal for relieving the scalp from dandruff and itching, and thus help strengthen the hair roots.

Regardless of the function of the insoluble particles, suspending them stably and homogeneously in the composition is a prerequisite for obtaining easy-to-use and visually appealing cosmetic compositions, as sought by consumers. However, obtaining cosmetic compositions comprising insoluble particles suspended homogeneously and stably therein (known as “smooth” cosmetic compositions) proves difficult, because these particles tend not to stay suspended, but to accumulate at the bottom of the containers containing the cosmetic compositions, especially as the stabilization of the suspension of insoluble particles must be carried out without impairing the cleansing and foaming power of the composition.

Furthermore, the viscosity of the cosmetic compositions must be adapted, by the choice of suitable constituents, to the desired packaging and dispensing method. The cosmetic compositions can be packaged in tubes or pump bottles, which results in constraints in terms of their viscosity, an overly thick formula not being “pumpable”, for example. In addition, an overly fluid formula will slip away from the consumer's hands, and is not desirable, as it does not match the required organoleptic quality for hygiene products.

Finally, it is highly desirable that cosmetic compositions comprise as little as possible of, and preferably be free from, any controversial substances, because consumers are increasingly careful regarding the composition of their cosmetic products. Thus, they seek products that are as free from ingredients obtained by chemical synthesis as possible, preferring ingredients of natural origin, known for their better tolerance and affinity with the skin, and which are more environmentally friendly.

In this regard, many cosmetic compositions for body and hair applications proposed by the prior art comprise sulfated surfactants. These surfactants ensure a particularly interesting foaming effect, giving the impression of gentle washing. However, in reality, they are very irritating for the skin, as they are drying and allergenic. Similarly, the presence of some amphoteric surfactants such as amidopropyl betaines, and in particular cocamidopropyl betaine, is suspected to cause some intolerances such as skin irritations. Thus, for good skin tolerance, these ingredients must be used at low concentrations, still with a risk when targeting consumers with particularly pronounced skin sensitivity. It is hence preferable to avoid these ingredients completely. The use as proposed by the prior art of acrylate-based suspending agents, such as Carbopol® 1382 (INCI name: Acrylates/C10-30 Alkyl Acrylate Crosspolymer) is furthermore also criticized. Indeed, these acrylates would be the cause of some allergic skin reactions, rhino-conjunctivitis and even asthma.

The document “Mild and Natural Shower Scrub” (Anonymous, Nov. 25, 2019, available, subject to having a user account, at the following URL: https://www.ulprospector.com/documents/1599626.pdf?bs=77&b=1461460&st=20&r=eu&ind=personalcare) describes an exfoliating shower scrub composition based on exfoliating apricot kernel powder, containing water, a surfactant mixture and the proprietary product Arbalon™ R-50. The surfactant mixture consists of sodium lauroyl methyl isethionate, cocamidopropyl betaine and coco glucoside (INCI names).

The document “Moisturizing Flaky-free Scalp Shampoo” (Anonymous, Jan. 27, 2022, available, subject to having a user account, at the following URL: https://www.ulprospector.com/documents/1625184.pdf?bs=77&b=5268232&st=20&r=eu&ind=personalcare) describes a shampoo composition containing water, a surfactant mixture, and the proprietary product Arbalon™ R-50 as a rheology modifier. The surfactant mixture consists of disodium cocamphodiacetate and cocamidopropyl hydroxysultaine (INCI names). This composition is free from insoluble molecules.

The document “SPA & Salon at Home Creamy Shampoo” (Anonymous, Oct. 22, 2021, available, subject to having a user account, at the following URL: https://www.ulprospector.com/documents/1625185.pdf?bs=77&b=5268233&st=20&r=eu&ind=personalcare) describes a shampoo composition based on insoluble glycol distearate particles. This composition contains water, a surfactant mixture and the proprietary product Arbalon™ R-50. The surfactant mixture consists of sodium lauroyl sarcosinate, cocamidopropyl betaine and lauryl glucoside (INCI names).

The document “Arbalon™ R-50 cellulose liquid” (Anonymous, Oct. 27, 2021, available, subject to having a user account, at the following URL: https://www.ulprospector.com/documents/1598326.pdf?bs=77&b=1423123&st=20&r=eu&ind=personalcare), technical data sheet of the commercial proprietary product Arbalon™ R-50, indicates, in a general way, that this proprietary product can be used to impart a real yield strength to shower gel and shampoo type cosmetic formulations, and suspend insoluble particles.

Document US 2013/143784 describes a hair cleansing composition comprising, in water, a surfactant mixture including at least one alkyl poly glucoside, a compound comprising a betaine group and at least one amino acid acyl. This composition is free from insoluble particles. In its introduction, this document states that it is desirable that the composition be free from substances such as cellulose and its derivatives.

Achieving a stable suspension of insoluble particles in a cosmetic composition for skin and/or hair hygiene and/or care, without negatively impacting the efficacy, nor the final viscosity, of this composition, and without altering the natural aspect of the formula, is a real challenge. None of the cosmetic compositions proposed to date by the prior art meets this need in a satisfactory manner.

The present invention aims to solve the drawbacks of these cosmetic compositions of the prior art, and to provide a smooth-looking, fluid cosmetic composition containing insoluble particles that are kept suspended therein in a stable manner, which has a good washing efficacy and a high foaming power, while being free from non-irritating or otherwise harmful ingredients for the skin, and which respects the epidermis and therefore poses the lowest possible risk of skin intolerance.

Additional objectives of the invention are that this composition contains the fewest possible ingredients that are not of natural origin, and preferably no such ingredients, that it has a physiological pH, and that it be suitable for use on all skin types, including sensitive skins and atopic skins.

More generally, the invention aims to ensure that this composition contains the fewest possible ingredients.

Another aim of the invention is that this composition has a sufficient viscosity so as not to slip away from the user's hand when handled and to allow its packaging in various types of packaging and dispensing devices, in tubes and pump bottles.

### DISCLOSURE OF THE INVENTION

It has been discovered by the present inventors that these objectives are achieved by a fluid cosmetic composition comprising, in an acceptable aqueous cosmetic vehicle, insoluble particles, a particular combination of surfactants, and a mixture of cellulose and cellulose gum, this mixture making it possible to effectively and lastingly keep the particles in a homogeneously suspended state in the vehicle.

Thus, according to a first aspect, the present invention proposes a cosmetic composition, particularly suitable for a topical application, in particular on the skin and hair, which contains:
a surfactant mixture comprising, in particular consisting of:
   a/ at least one anionic surfactant selected from among alaninates,
   b/ by way of non-ionic surfactant(s), lauryl glucoside optionally in a mixture with at least one other non-ionic surfactant chosen from alkyl glucosides wherein the alkyl radical comprises 8 to 16 carbon atoms,
   c/ at least one amphoteric surfactant different from amidopropyl betaines,
a mixture of cellulose and cellulose gum, in a content between a value strictly greater than 0.05% and a value less than or equal to 0.5%, by weight relative to the total weight of the composition,
insoluble particles in stable suspension in the composition,
and water.

This composition is furthermore free from amphoteric surfactant of the class of amidopropyl betaines and from surfactant comprising at least one sulfate group. It furthermore preferably comprises at most 0.5% by weight of gum(s) other than cellulose gum, relative to the total weight of the composition. This means that it is free from gum(s) other than cellulose gum, or, when it comprises one or more gums other than cellulose gum, the total concentration thereof in the composition is less than or equal to 0.5% by weight relative to the total weight of the composition.

In the present description, the term “cosmetically acceptable” means that the substance is suitable for use by placing in contact with human and animal cells, in particular skin or hair cells. Preferably, this substance has an agreeable odor, color and feel, and does not generate unacceptable discomforts which could deter a user from the composition according to the invention.

The term insoluble particles means particles that have no solubility in the vehicle of the composition, including when mixed with the other ingredients thereof. Thus, the insoluble particles are in solid form in the composition.

The term stable suspension means, conventionally per se, that the particles do not settle or aggregate. Thus, virtually no settling of the insoluble particles contained therein is observed in the composition according to the invention, including for long periods, in particular for a period as long as the general shelf-life of the composition, which is more than 6 months, preferably more than 12 months, more preferably more than 18 months and preferentially 36 months. In particular, this stability can be checked by visual observation of the composition. Preferably, the insoluble particles are kept dispersed in the composition in a substantially homogeneous manner.

The cosmetic composition according to the invention can thus be described as a smooth composition, this smooth appearance, linked with the very good stability of the suspension of the insoluble particles in the vehicle, being advantageously obtained by the joint use of cellulose and cellulose gum, natural ingredients which, in addition to their suspending effect, do not negatively impact the synergistic effect of the different surfactants included in the composition and give it a particularly high foaming power, and a good foam quality.

The cosmetic composition according to the invention furthermore poses a low skin irritation risk, lower than that of the cosmetic compositions of the prior art. It also has a good viscosity, even without the need to use an additional ingredient having a thickening effect. In the present description, the term “good viscosity” means a viscosity enabling the composition to be dispensed by various packagings, in particular tubes and pump bottles, and enabling it not to slip away from the user's hands when it is dispensed therein. Thus, in preferred embodiments of the invention, the composition is free from additional ingredients having a thickening effect. It is in particular preferably free from alginates, carrageenans, starch, gums such as xanthan gum, diutan gum, gellan gum, sclerotic gum, succinoglycan gum, gum arabic, carob gum, Tamarindus indica seed gum, Caesalpinia spinosa gum, etc. The composition according to the invention is furthermore preferably free from ethylene glycol polymers (PEG) and their derivatives, which have a negative environmental impact.

Furthermore, the composition according to the invention is substantially free from an oily phase. Thus, it is not an emulsion, or a biphasic composition, but rather a monophasic fluid composition. It is thus in particular substantially free from fatty acids. This means that it contains no fatty acids, or in a concentration of less than 0.05%, preferably 0.01%, by weight. It likewise preferably contains no emulsifiers such as a fatty acid monoester of polyethylene glycol.

In particular embodiments of the invention, the cosmetic composition comprises at least 50% by weight of water relative to the total weight of the composition. Preferably, the composition according to the invention comprises at least 55%, more preferably at least 60%, and preferentially at least 65%, by weight of water relative to the total weight of the composition.

In the present description, the term “at least one” means a single compound or a mixture of several such compounds. Thus, the surfactant mixture of the composition according to the invention contains, and preferably consists of:
a/ a single anionic surfactant selected from among alaninates, or a plurality of such anionic surfactants;
b/ a single non-ionic surfactant that is lauryl glucoside, or a plurality of non-ionic surfactants including lauryl glucoside as well as another or a plurality of other non-ionic surfactants chosen from alkyl glucosides the alkyl radical of which comprises 8 to 16 carbon atoms; this or these non-ionic surfactant(s) may optionally be in a mixture with one or more other non-ionic surfactants, such as for example C8-C16 fatty alcohol glucosides, for example coco glucoside;
c/ and a single amphoteric surfactant different from amidopropyl betaines, or a plurality of amphoteric surfactants different from amidopropyl betaines.

The anionic surfactant(s) selected from among alaninates can be formed by any cosmetically acceptable counterion. The counterion is preferably sodium.

In particular embodiments of the present invention, the anionic surfactant(s) selected from among alaninates are preferably chosen from sodium cocoyl alaninate (INCI name) and trisodium dicarboxymethyl alaninate (INCI name). Preferably, the composition according to the invention comprises no other anionic surfactant that is not an alaninate. It comprises for example a single anionic surfactant, sodium cocoyl alaninate.

In particular embodiments of the present invention, the composition comprises no other non-ionic surfactant that is not an alkyl glucoside wherein the alkyl radical comprises 8 to 16 carbon atoms. It comprises for example a single non-ionic surfactant, lauryl glucoside.

In other particular embodiments, the composition comprises at least one second non-ionic surfactant chosen from alkyl glucosides wherein the alkyl radical comprises 8 to 16 carbon atoms. The alkyl glucosides are preferably chosen from the compounds with the INCI names: octyl glucoside and decyl glucoside.

In particular embodiments of the present invention, the amphoteric surfactant(s) different from amidopropyl betaines are chosen from substances with the INCI names: Sodium Caproamphoacetate, Sodium Caproamphopropionate, Sodium Apryloamphoacetate, Sodium Caprylamphopropionate, Sodium Cocoamphoacetate, Sodium Cocoamphopropionate, Sodium Isostearoamphoacetate, Sodium isostearoamphopropionate, Sodium Lauroamphoacetate, Sodium Lauroamphopropionate, Sodium Oleoamphoacetate, Sodium Oleoamphopropionate, Sodium Stearoamphoacetate, Sodium Stearoamphopropionate, Sodium Tallowamphoacetate, Sodium Wheatgermamphoacetate, Disodium Caproamphodiacetate, Disodium Caproamphodipropionate, Disodium Capryloamphodiacetate, Disodium Cocoamphodiacetate, Disodium Isostearoamphodiacetate, Disodium Lauroamphodiacetate, Disodium Oleoamphodipropionate, Disodium Capryloamphodipropionate, Disodium Cocoamphodipropionate, Disodium Isostearoamphodipropionate, Disodium Lauroamphodipropionate, Disodium Stearoamphodiacetate, Disodium Tallowamphodiacetate and Disodium Wheatgermamphodiacetate.

Preferably, said at least one amphoteric surfactant different from amidopropyl betaines is chosen from lauryl betaine, coco betaine, sodium cocoamphoacetate and disodium cocoamphodiacetate, where these compounds can be used in the composition alone or in any of the mixtures thereof.

In particular embodiments of the invention, the surfactant mixture contains a single amphoteric surfactant which is coco betaine.

In particular embodiments of the invention, the composition is free from:
any surfactant of the type having an isethionic acid or isethionate group, in particular surfactant chosen from acyl isethionic acids or salts thereof, such as the substance with the INCI name sodium cocoyl isethionate;
and/or any surfactant of the type having a N-methyltaurine or taurate group, in particular surfactant chosen from N-acyltaurines or salts thereof, such as the substance with the INCI name sodium methyl cocoyl taurate;
and/or any surfactant of the type having a sulfosuccinic acid or sulfosuccinate group, in particular surfactant chosen from alkyl sulfosuccinic acids or salts thereof, such as the substance with the INCI name lauryl sulfosuccinate;
and/or any surfactant comprising at least one sulfate group, i.e. a —O—SO₃H or —SO₂—O-group, such as laureth sulfate or lauryl sulfate.

Without altering the performance of the composition, this advantageously reduces the presence within said composition of compounds conventionally used in cosmetic compositions for skin and hair hygiene or care which have the disadvantage of a non-natural origin.

Moreover, although sulfated surfactants have a particularly interesting foaming power, they are aggressive for the epidermis. Thus, their absence in the composition according to the present invention advantageously reduces the risks of aggression of the user's epidermis.

Preferably, the composition according to the invention comprises no other surfactant than: one or more alaninates, one or more alkyl glucosides the alkyl radical of which comprises 8 to 16 carbon atoms, including at least lauryl glucoside, and one or more amphoteric surfactants different from amidopropyl betaines.

Generally, the composition according to the invention preferably contains the fewest possible surfactants. Thus, in particular embodiments of the invention, the composition comprises not more than 6 surfactants, preferably not more than 5 surfactants, more preferably not more than 4 surfactants and it preferably contains only 3 surfactants. As stated above, a high foaming power as well as a good quality of the foam formed can be obtained with a surfactant mixture comprising a single compound of each of three defined surfactant families, namely: an anionic surfactant selected from among alaninates, the non-ionic surfactant lauryl glucoside and an amphoteric surfactant different from amidopropyl betaines. This advantageously makes it possible to minimize the number of ingredients present in the composition and thus reduce the manufacturing costs thereof.

In preferred embodiments of the invention, the content of said surfactant mixture in the composition is comprised between 5 and 18% by weight, preferably between 6 and 16% by weight, more preferably between 8 and 15% by weight, relative to the total weight of the composition. Such ranges of values make it possible to obtain a good foaming power of the composition, a good quality of foam formed and a good viscosity of the composition, while being associated with a reduced manufacturing cost of the composition.

The total content of anionic surfactant(s) selected from among alaninates is preferably between 1.5 and 5% by weight, more preferably between 2 and 4% by weight, relative to the total weight of the composition.

In the particular embodiments of the invention wherein the composition comprises a single anionic surfactant, preferably sodium cocoyl alaninate, the latter is thus preferably present in the composition in a content of between 1.5 and 5% by weight, more preferably between 2 and 4% by weight, in particular between 3 and 4% by weight, relative to the total weight of the composition. The total content of non-ionic surfactant(s) is preferably comprised between 3 and 10% by weight, more preferably between 5 and 8% by weight, in particular between 5 and 7% by weight, relative to the total weight of the composition.

Thus, the total content of lauryl glucoside as the only non-ionic surfactant of the composition is preferably comprised between 3 and 10% by weight, more preferably between 5 and 8% by weight, in particular between 5 and 7% by weight, relative to the total weight of the composition.

In particular embodiments of the invention, the total content of non-ionic surfactants consisting of lauryl glucoside accompanied by at least one non-ionic surfactant chosen from alkyl glucosides wherein the alkyl radical comprises 8 to 16 carbon atoms is preferably comprised between 3 and 10% by weight, more preferably between 5 and 8% by weight, in particular between 5 and 7% by weight, relative to the total weight of the composition.

The total content of said at least one amphoteric surfactant different from amidopropyl betaines is preferably comprised between 0.6 and 3% by weight, more preferably between 1.5 and 2.5% by weight, in particular between 1.5 and 2% by weight, relative to the total weight of the composition.

In the particular embodiments of the invention wherein the composition contains a single amphoteric surfactant, preferably coco betaine, the latter is thus preferably present in the composition in a content of between 0.5 and 3% by weight, preferably between 1.5 and 2.5% by weight, in particular between 1.5 and 2% by weight, relative to the total weight of the composition.

In particular embodiments of the invention, the mixture of cellulose and cellulose gum comprises substantially equal mass quantities of cellulose and cellulose gum.

The cellulose and the cellulose gum, also known as carboxymethylcellulose, can come from any origin, in particular from a microbial origin and/or a plant origin. The cellulose is preferably in microcrystalline form, for example complying with CAS No. 9004-34-6.

The cellulose gum is preferably sodium carboxymethylcellulose, of CAS No. 9004-32-4.

In particular embodiments of the invention, the mixture of cellulose and cellulose gum is in liquid form.

By way of example, the mixture of cellulose and cellulose gum can be such as marketed under the name Arbalon® R-50, which furthermore has the advantage of containing glycerin, a compound with particularly advantageous emollient properties in the context of the field of topical cosmetic compositions.

The total content of said mixture of cellulose and cellulose gum in the composition is preferably comprised between a value strictly greater than 0.05%, in particular greater than or equal to 0.1%, and a value less than or equal to 1%, preferably less than or equal to 0.9%, more preferably less than or equal to 0.5%, and preferentially less than or equal to 0.45%, by weight, relative to the total weight of the composition. The content of the mixture of cellulose and cellulose gum in the composition is comprised between a value strictly greater than 0.05% and a value less than or equal to 0.5% by weight, preferably less than or equal to 0.45% by weight, relative to the total weight of the composition. The content of the mixture of cellulose and cellulose gum in the composition can be between 0.1 and 0.5% by weight, in particular between 0.1 and 0.45% by weight, more preferably between 0.15 and 0.5% by weight, in particular between 0.15 and 0.45% by weight, and preferentially between 0.15 and 0.3% by weight, relative to the total weight of the composition.

In particular embodiments of the invention, the insoluble particles comprise pearlescent agents, exfoliating particles and/or millicapsules.

Pearlescent agents are ingredients commonly used in cosmetic formulations, as agents providing a pearly appearance to the composition wherein they are incorporated. Mention can be made in this regard of glycol distearate or ethylene glycol distearate.

“Exfoliating particles” or “scrubbing particles” mean solid and abrasive particles which are in particular capable of performing, when they are applied by massage on the skin comprised in a cosmetic composition, a mechanical exfoliation (also called scrub) of the dead cells of the superficial layers of the epidermis. Generally, a skin scrub is typically performed by applying a composition comprising exfoliating particles on the skin, massaging the skin coated with said composition, then rinsing said composition. The exfoliating particles then act by rubbing on the skin. The skin is thus smoothed, and cell renewal thus promoted. According to the present invention, the exfoliating particles preferably have a median size between 10 and 1500 μm, more preferably between 50 and 1000 μm, and even more preferably between 200 and 500 μm, for example between 250 and 450 μm. “Median size” of the exfoliating particles herein means the volume median diameter. The median size of the exfoliating particles can be measured by static light scattering using a Mastersizer type particle size analyzer.

According to the invention, the following can be used as exfoliating particles:
exfoliating particles of mineral origin, such as sands, rocks, perlite or clays;
exfoliating particles of plant origin, such as kernels, leaves, stalks, algae, bark, nuts or roots;
and/or exfoliating particles of organic origin, such as polyethylene- or wax-based beads.

For example, polyethylene beads or powder, perlite beads or powder, polyvinyl chloride powder, pumice stone (INCI name: pumice), crushed fruit kernel shells such as ground apricot kernels or walnut shells, sawdust, wood flour, cork flour, glass beads, alumina (aluminum oxide), sugar crystals (sucrose), etc.; or any of the mixtures thereof, can be used.

In the present description, the term millicapsules means generally substantially spherical entities, of millimetric size, typically between 10 and 2000 μm, formed from a destructible, in particular breakable, in particular solid and in particular continuous, shell, enclosing a core comprising one or more ingredients.

“Millicapsules” means spherical aggregates with a diameter ranging from about 10 to 2000 μm, which contain at least one solid or liquid core which is enclosed by at least one continuous shell.

The millicapsules used according to the present invention can contain any active ingredient or a mixture of active ingredients that can be of interest to release during application, in particular on the skin and/or hair, of the cosmetic composition according to the invention, and that the coating in the capsule protects throughout the shelf-life of the composition, in particular from pro-oxidant elements such as free radicals, oxygen or ultraviolet rays.

The active substance content, or encapsulation rate, can be very high, in particular in the order of 85 to 90%, expressed as a percentage by weight of active ingredient(s) relative to the total weight of the capsule.

The active ingredients can in particular include fragrances, essential oils, vitamins, etc.

The shell can for example be formed from polymer(s) of marine or plant origin, from semi-synthetic material or from synthetic material. It can for example be formed from gum arabic, agar agar, agarose, maltodextrin, alginic acid or one of the salts thereof, for example sodium or calcium alginate, from fat, fatty acid, cetyl alcohol, collagen, chitosan, lecithin, gelatin, albumin, shellac, polysaccharide such as dextran or starch and its derivatives, in particular starch ethers and esters, from polypeptide, protein hydrolysate, sucrose, wax, etc.; or from chemically modified cellulose, in particular cellulose ester or ether, for example from cellulose acetate, ethylcellulose, hydroxypropylcellulose, hydroxypropylmethylcellulose or carboxymethylcellulose; or from polymer such as polyacrylates, polyamides, polyvinyl alcohol or polyvinylpyrrolidone.

The millicapsules used in the composition according to the invention can be obtained in any conventional manner per se, for example by coating liquid or finely dispersed solid phases in a film-forming polymer, the latter precipitating on the material to be coated after emulsification and coacervation or interfacial polymerization.

The millicapsules used according to the invention can be free from any pigment or colorant, or be colored, for example contain, in their core, their shell or in both, one or more colored compounds.

The insoluble particle content in the composition according to the invention is variable according to the nature of the insoluble particle.

Thus, when the insoluble particles comprise one or more pearlescent agents, in particular are composed of one or more pearlescent agents, the content of such pearlescent agents, for example glycol distearate, is preferably comprised between 0.1 and 1.2% by weight, preferably between 0.5 and 1% by weight, in particular about 0.8% by weight, relative to the total weight of the composition.

When the insoluble particles comprise exfoliating particles, in particular consist of exfoliating particles, the exfoliating particle content is between 1 and 2.5% by weight, preferably between 1.5 and 2.2% by weight, in particular of about 2% by weight, relative to the total weight of the composition.

When the insoluble particles comprise millicapsules, in particular consist of millicapsules, the millicapsule content is between 1 and 2% by weight, preferably between 1 and 1.5% by weight, in particular of about 1.2% by weight, relative to the total weight of the composition.

The cosmetic composition according to the invention contains in particular, the contents being expressed relative to the total weight of the composition:
at least 50% by weight of water;
5 to 18% of a surfactant mixture consisting of:
   a/ 1.5 to 5% by weight, in particular between 3 and 4% by weight, of one or more anionic surfactant(s) selected from among alaninates, such as sodium cocoyl alaninate,
   b/ 3 to 10%, in particular between 5 and 7% by weight, of lauryl glucoside alone or in a mixture with at least one other non-ionic surfactant chosen from alkyl glucosides the alkyl radical of which comprises 8 to 16 carbon atoms,
   c/ 0.5 to 3%, in particular between 1.5 and 2% by weight, of at least one amphoteric surfactant different from amidopropyl betaines, such as coco betaine;
0.1 to 0.5% by weight, in particular 0.3 to 0.45% by weight, of a mixture of cellulose and cellulose gum, preferably in substantially equal respective quantities by weight;
and insoluble particles in stable suspension in the composition, in particular 0.1 to 2% by weight of pearlescent agent(s) particles and/or 1 to 2.5% by weight of exfoliating particles and/or 1 to 2% by weight of millicapsules.

The cosmetic composition according to the invention is preferably free from ethylenediaminetetraacetic acid (EDTA).

The cosmetic composition according to the invention can further comprise at least one, i.e. one or more, cosmetically acceptable excipient(s), preferably chosen from emollients, conditioning agents, solubilizers, fragrances, and mixtures thereof.

In particular embodiments of the present invention, the cosmetic composition contains one or more emollients, in particular chosen from glycerin, diglycerin or sorbitol.

In particular embodiments of the present invention, the cosmetic composition contains one or more conditioning agents, in particular chosen from gums, other than cellulose gum, in particular guar gum and its derivatives, polyquaterniums, polyglyceryls, polysaccharides, squalane etc. Preferably, the conditioning agent is guar gum or one of its derivatives. According to preferred embodiments of the present invention, the total conditioning agent(s) content is comprised between 0.1 and 1% by weight relative to the total weight of the composition. The total content of conditioning agent(s) chosen from gums other than cellulose gum, such as guar gum or its derivatives, is less than or equal to 0.5% by weight relative to the total weight of the composition.

In particular embodiments of the present invention, the cosmetic composition according to the invention contains one or more solubilizers, in particular chosen from the polyglyceryl family, such as polyglyceryl-4 caprate, polyglyceryl-6 caprylate, polyglyceryl-3 cocoate, polyglyceryl-6 ricinoleate, glyceryl oleate, coco-glucoside.

As stated above, in particular embodiments of the invention, the cosmetic composition is free from additional ingredients having a thickening effect, in order to minimize the number of ingredients in the formulas. As explained above, all of the constituents of the composition, in particular the surfactant mixture, advantageously give the composition a good viscosity and in particular a viscosity allowing it to be free from additional ingredients having a thickening effect, even when it comprises one or more cosmetically acceptable excipients such as emollients, conditioning agents, solubilizers and fragrances. The number of ingredients of the composition is thus reduced and the associated manufacturing costs are therefore likewise reduced.

In other particular embodiments of the present invention, the composition nevertheless comprises at least one additional ingredient having a thickening effect.

The additional ingredient with a thickening effect is then preferably chosen from alginates, carrageenans, starch and gums, other than cellulose gum, such as diutan gum, gellan gum, sclerotic gum, succinoglucan gum, gum arabic, carob gum, Tamarindus indica seed gum, Caesalpinia spinosa gum, etc. The total content of such additional ingredient(s) chosen from gums other than cellulose gum is less than or equal to 0.5% by weight relative to the total weight of the composition.

In particular embodiments of the present invention, the cosmetic composition contains one or more cosmetically active agents, in particular by topical application, for example chosen from hydrating agents, antioxidants, vitamins, etc.

The total content of cosmetically active ingredient(s) in the cosmetic composition according to the invention is preferably comprised between 0.05 and 3% by weight relative to the total weight of the composition.

In particular embodiments of the invention, the cosmetic composition comprises at least one, i.e. one or more, cosmetically active agent(s), chosen from vitamins, hyaluronic acid, oat sap, helychrysum extracts, helychrysum essential oils, limnanthes oil, in particular Limnanthes alba seed oil, oat seedlings, flower acids, such as the product marketed under the name Flower Acids®, garcinia extracts, milk thistle oil, niacinamide, edelweiss extracts, such as the product marketed under the name Alpaflor® Edelweiss, cinchona extracts, caffeine, peony extracts, mango butter and citron extracts, such a list not limiting the invention in any way.

The cosmetic composition according to the invention preferably has a dynamic viscosity at 22° C.+/−2° C. greater than 700 mPa·s, preferably between 1500 and 4000 mPa·s. This viscosity can for example be measured by means of a rotary rheometer, such as the Rheomat RM200 (module M2), at a shear rate of 17.7 s⁻¹, for a shear time of 10 s.

According to the type of insoluble particles contained therein, the cosmetic composition according to the invention can be transparent or opaque. A transparent composition is defined in the present description, conventionally, as a composition having a turbidity of less than 20 NTU (Nephelometric Turbidity Units). An opaque composition is defined in the present description, conventionally, as a composition having a turbidity greater than 1000 NTU. The turbidity can be measured using a HACH TL2310 type turbidity meter. Before each turbidity measurement, the correct adjustment of the instrument is preferably checked by measuring the turbidity of two standards from a StabCal 0 to 4000 NTU standard calibration kit, the standards being chosen so that they frame the expected value of the sample.

The cosmetic composition according to the invention, in the desired form, can be prepared with any conventional method per se for the preparation of cosmetic compositions. A particularly preferred preparation method within the scope of the invention comprises:
in an acceptable cosmetic vehicle, in particular water, adjusting the pH to 4, for example with citric acid;
under stirring, adding the mixture of cellulose and cellulose gum, as well as insoluble particles, and stirring for example for 20 minutes at 3000 rpm, so as to obtain an opaque translucent appearance;
very rapid deaeration, e.g. using a vacuum chamber;
slowly re-homogenizing, for example for 5 min, in particular in a deflocculating turbine with holes at 250 rpm, so as to resuspend the cellulose and the cellulose gum and to allow a suspension of the particles homogeneously in the formula;
introducing, at low stirring speed, for example 450 rpm, for example in a deflocculating turbine with holes at 450 rpm, the amphoteric surfactant(s), followed by the non-ionic surfactant(s), previously incubated at 40° C., followed by the anionic surfactant(s); the formula becomes transparent;
optionally, adding sodium benzoate;
stirring for about 20 minutes, for example at 450 rpm, until a homogeneous mixture is obtained;
optionally, adjusting the pH with citric acid then stirring at 450 rpm for 5 to 15 minutes.

In the particular embodiments wherein the cosmetic composition comprises at least one conditioning agent, such as a guar gum derivative, the latter is preferably introduced under vigorous stirring into the water in the initial step of the method.

The cosmetic composition according to the present invention can be used on all keratin materials, such as the skin, scalp, hair, eyelashes, eyebrows, nails or mucous membranes, in particular packaged in the form of a hygiene product, for example in the form of a product for cleansing the skin, mucous membranes and/or hair, in particular in the form of a product for cleansing the skin and/or removing make-up from the skin, in particular of the face and/or the body, in the form of a shower product, in the form of a shampoo, etc.

It can in particular be in the form of a shower gel, shampoo or face and/or body foaming gel.

Another aspect of the invention relates to the non-therapeutic cosmetic use of a cosmetic composition according to the invention for washing and/or care of the skin, in particular the skin of the body and/or face and/or scalp, and/or the hair, of an individual, preferably by topical application.

This use preferably comprises the successive steps consisting of:
applying the composition on the skin and/or hair, the latter having preferably been made wet beforehand,
developing foam, in particular by rubbing the skin and/or hair on which the composition has been applied,
and rinsing, preferably with water, optionally after a resting time and/or an optional massage.

These steps can, for example, be implemented once or twice per day, for example morning and evening, or as often as necessary.

The composition according to the invention can, in particular, be used for washing and/or the care of sensitive or atopic skin.

In the present description, sensitive skin means a skin that is prone to irritations or allergic reactions because of various internal and external, non-immunological factors. The term atopic skin means a skin having an inflammatory disease characterized by a dryness of the skin associated with itching as in the case of eczema or atopic dermatitis.

The invention also relates to a non-therapeutic cosmetic treatment method for hygiene and/or care of the skin and/or hair of an individual, by means of a cosmetic composition according to the invention. This method comprises the successive steps consisting of:
applying the composition on the skin and/or hair, the latter having preferably been made wet beforehand,
developing foam by rubbing the composition on the skin and/or the hair,
and rinsing, preferably with water.

This method can comply with any feature, or combination of features, described above with reference to the cosmetic use of the composition according to the invention.

The features and advantages of the invention will become more apparent in light of the examples of implementations below, provided purely by way of illustrative and non-limiting examples of the invention.

### EXAMPLES

### 1/Example 1: Evaluation of Different Compounds with Suspending Properties

The suspension of insoluble particles was tested for several compounds with suspending properties in formulas that are otherwise according to the invention and transparent, which allows to quickly visually detect a homogeneous distribution of the insoluble particles.

For this evaluation, the formulas included, in % by weight relative to the total weight of the formula:
sodium cocoyl alaninate (3-4% by weight)
lauryl glucoside (5-7% by weight),
coco betaine (1.5-2% by weight)
insoluble particles: millicapsules (1.2% by weight),
compound under test (according to % by weight indicated in Table 1),
water qs 100%.

The insoluble particles are initially suspended homogeneously in the formulas, by stirring.

The compositions were evaluated according to the following criteria:
stability over time of the suspension of insoluble particles,
visual appearance (transparency, homogeneity).

The results obtained, in terms of visual appearance of the formula after about 15 min, are given in Table 1 for each compound tested.

TABLE 1

| Compound(s) (% by weight) | Appearance of formula |
|---|---|
| 50/50 mixture of cellulose | Transparent, smooth |
| and cellulose gum (0.2%) | |
| Xanthan gum (0.8%) | Cloudy-opaque, jelly appearance |
| Sodium alginate (1%) | Cloudy, inhomogeneous appearance |
| Carrageenate (0.5%) | Cloudy, inhomogeneous appearance |
| Cellulose gum (1%) | Cloudy and granular |
| Diutan gum (0.1%) | Cloudy, phase separation |
| 50/50 mixture of cellulose | Opaque-viscous and granular, |
| and cellulose gum (0.2%) | jelly appearance |
| and xanthan gum (0.7%) | |

It appears that, in this surfactant mixture according to the invention, the transparency and stability of the insoluble particles are only satisfactory in the presence of the mixture of cellulose and cellulose gum according to the invention. For the other compound(s), the cloudy appearance is observed very quickly, in the first minutes after composition preparation.

### 2/Example 2: Evaluation of the Range of Use of the Mixture of Cellulose and Cellulose Gum

For this evaluation, the formulas comprise, relative to the total weight of the formula:
sodium cocoyl alaninate (3-4% by weight),
lauryl glucoside (5-7% by weight),
coco betaine (1.5-2% by weight),
insoluble particles (type and % by weight below),
50/50 mixture of cellulose and cellulose gum (according to % by weight below),
water qs 100%.

The insoluble particles are initially suspended homogeneously in the formulas, by stirring.

The visual appearance makes it possible to validate or invalidate, whether the composition has a homogeneous appearance, the insoluble particles being kept in stable suspension in the formula.

In the case where the insoluble particles are millicapsules at 1.2% by weight, a concentration of 0.3% by weight of the 50/50 mixture of cellulose and cellulose gum was tested. It is observed that the millicapsules remain lastingly homogeneously distributed in suspension in the composition.

In the case where the insoluble particles are pearlescent agents, more specifically glycol distearate particles, at a concentration of 0.8% or 1.2% by weight, the results obtained, in terms of visual appearance of the formula, are shown in Table 2.

TABLE 2

| | 50/50 mixture of cellulose and cellulose | | |
|---|---|---|---|
| Pearlescent agent | gum content (% by weight) | | |
| content (% by weight) | 0.05% | 0.15% | 0.3% |
| 0.8% | / | ++ | ++ |
| 1.2% | − | ++ | ++ |

(“−”: the particles do not stay homogeneously distributed in the composition, and fall rapidly); “++”: the particles stay lastingly homogeneously suspended in the composition)

These tests show that 0.05% of the mixture of cellulose and cellulose gum is insufficient to homogeneously suspend 1.2% of the pearlescent agent. On the other hand, for 0.15% of this mixture and over, the pearlescent agent particles appear suspended in the composition homogeneously and lastingly over time.

### 3/Example 3: Examples of Compositions According to the Invention

A cosmetic composition according to the invention with homogeneously distributed pearlescent agent particles, intended for cosmetic treatment, for hair hygiene and care, complies with the features listed in Table 3 below:

TABLE 3
Example of opaque composition according to
the invention for hair hygiene and care

| Ingredients (INCI name) | % w/w |
|---|---|
| Sodium cocoyl alaninate | 3-4 |
| Lauryl glucoside | 5-7 |
| Coco betaine | 1.5-2.0 |
| Water | Qs 100 |
| Cellulose/cellulose gum | 0.15 |
| Glycol distearate | 0.8 |
| Guar | 0.3 |
| Glycerin | 0.38 |

Another example of a composition according to the invention containing homogeneously distributed millicapsules, intended for cosmetic treatment, for hair hygiene and care, complies with the features listed in Table 4 below:

TABLE 4
Example of transparent composition according
to the invention for hair hygiene and care

| Ingredients (INCI name) | % w/w |
|---|---|
| Sodium cocoyl alaninate | 3-4 |
| Lauryl glucoside | 5-7 |
| Coco betaine | 1.5-2.0 |
| Water | Qs 100 |
| Cellulose/cellulose gum | 0.3 |
| Millicapsules | 1.2 |
| Guar | 0.3 |

Another example of a composition according to the invention containing homogeneously distributed pearlescent agent particles, intended for cosmetic treatment, for facial and/or body skin hygiene and care, is given in Table 5 below:

TABLE 5
Example of opaque composition, according to the
invention for facial and/or body hygiene and care

| Ingredients (INCI name) | % w/w |
|---|---|
| Sodium cocoyl alaninate | 3-4 |
| Lauryl glucoside | 5-7 |
| Coco betaine | 1.5-2.0 |
| Water | Qs 100 |
| Cellulose/cellulose gum | 0.15 |
| Glycol distearate | 0.8 |

Another example of a composition according to the invention containing homogeneously distributed millicapsules, intended for cosmetic treatment, for facial and/or body skin hygiene and care, is given in Table 6 below:

TABLE 6
Example of a transparent composition according to the
invention for facial and/or body skin hygiene and care

| Ingredients (INCI name) | % w/w |
|---|---|
| Sodium cocoyl alaninate | 3-4 |
| Lauryl glucoside | 5-7 |
| Coco betaine | 1.5-2.0 |
| Water | Qs 100 |
| Cellulose/cellulose gum | 0.3 |
| Millicapsules | 1.2 |
| Glycerin | 0.75 |

### 4/Example 4: Evaluation of the Features of the Compositions According to the Invention

The compositions according to the invention of the formula described in Table 7 below are evaluated on the following criteria:
foaming power (quantity/quality): foam height,
viscosity,
pH (measured with a pH meter),
stability of the particle suspension over time (visual observation).

For this purpose, the following operating protocols are implemented.

For the evaluation of the foam height, a 250-ml graduated measuring cylinder, of outer diameter 38.9 cm and height 36 cm, with a stopper, is used. 0.5 g of composition is placed in this cylinder and demineralized water is added to make up to 50 ml. The cylinder is stoppered then vigorously shaken 10 times. The foam height is read and expressed in mm.

The viscosity is measured using a Rheomat RM200 rotary rheometer (M2 module), at a shear rate of 17.7 s⁻¹, for a shear time of 10 s, at 22° C.+/−2° C. The results obtained with the different compositions (Comp. 1 to Comp. 4) are given in Table 7 below:

TABLE 7

| | Comp. 1 | Comp. 2 | Comp. 3 | Comp. 4 |
|---|---|---|---|---|
| Ingredients (INCI name) | % w/w | % w/w | % w/w | % w/w |
| Sodium cocoyl alaninate | 3-4 | 3-4 | 3-4 | 3-4 |
| Lauryl glucoside | 5-7 | 5-7 | 5-7 | 5-7 |
| Coco betaine | 1.5-2.0 | 1.5-2.0 | 1.5-2.0 | 1.5-2.0 |
| Water | Qs 100 | Qs 100 | Qs 100 | Qs 100 |
| Cellulose/cellulose gum | 0.3 | 0.3 | 0.3 | 0.3 |
| Guar | 0.3 | 0.3 | — | — |
| Millicapsules | — | 1.2 | — | 1.2 |
| Glycol distearate | 0.8 | — | 0.8 | — |
| Properties | | | | |
| Foam height (mm) | 200 | 200 | 200 | 220 |
| Viscosity (mPa · s) | 2738 | 4330 | 1022 | 1705 |
| pH | 5.35 | 5.33 | 5.26 | 5.40 |

It can be seen that the 4 compositions according to the invention have a very interesting foam height, and a satisfactory viscosity.

For the test duration of about 2 h, a good stability of the particle suspension is furthermore observed visually.

1. A cosmetic composition containing:
a surfactant mixture comprising:
a/ at least one anionic surfactant selected in the group consisting of alaninates,
b/ by way of non-ionic surfactant(s), lauryl glucoside optionally in a mixture with at least one other non-ionic surfactant chosen in the group consisting of alkyl glucosides wherein the alkyl radical comprises 8 to 16 carbon atoms,
c/ at least one amphoteric surfactant different from amidopropyl betaines,
a mixture of cellulose and cellulose gum, in a content between a value strictly greater than 0.05% and a value less than or equal to 0.5%, by weight relative to the total weight of the composition,
insoluble particles in stable suspension in said composition,
and water,
said composition being free from amphoteric surfactant of the class of amidopropyl betaines and from surfactant comprising at least one sulfate group,
and said composition comprising at most 0.5% by weight of gum(s) other than cellulose gum, relative to the total weight of the composition.

2. The cosmetic composition according to claim 1, comprising at least 50% by weight of water relative to the total weight of the composition.

3. The cosmetic composition according to claim 1, wherein said insoluble particles comprise pearlescent agents, present in said composition in a content of between 0.1 and 1.2% by weight relative to the total weight of the composition.

4. The cosmetic composition according to claim 1, wherein said insoluble particles comprise exfoliating particles, present in said composition in a content of between 1 and 2.5% by weight relative to the total weight of the composition.

5. The cosmetic composition according to claim 1, wherein said insoluble particles comprise millicapsules, present in said composition in a content of between 1 and 2% by weight relative to the total weight of the composition.

6. The cosmetic composition according to claim 1, wherein said mixture of cellulose and cellulose gum comprises equal mass quantities of cellulose and cellulose gum.

7. The cosmetic composition according to claim 1, wherein the content of said mixture of cellulose and cellulose gum is comprised between a value strictly greater than 0.05% and a value less than or equal to 0.45% by weight relative to the total weight of the composition.

8. The cosmetic composition according to claim 1, comprising at least one conditioning agent chosen in the group consisting of guar gum and its derivatives, polyquaterniums, polyglyceryls, polysaccharides and squalane.

9. The cosmetic composition according to claim 1, wherein the content of said surfactant mixture is comprised between 5 and 18% by weight relative to the total weight of the composition.

10. The cosmetic composition according to claim 1, wherein said surfactant mixture contains a single anionic surfactant which is sodium cocoyl alaninate, present in said composition in a content of between 1.5 and 5% by weight relative to the total weight of the composition.

11. The cosmetic composition according to claim 1, wherein said surfactant mixture contains a single amphoteric surfactant which is coco betaine, present in said composition in a content of between 0.5 and 3% by weight relative to the total weight of the composition.

12. The cosmetic composition according to claim 1, comprising at least one cosmetically active agent chosen in the group consisting of vitamins, hyaluronic acid, oat sap, helychrysum extracts, helychrysum essential oils, limnanthes oil, oat seedlings, flower acids, garcinia extracts, milk thistle oil, niacinamide, edelweiss extracts, cinchona extracts, caffeine, peony extracts, mango butter and citron extracts.

13. The cosmetic composition according to claim 1, in the form of shower gel, shampoo, face and/or body foaming gel.

14. The cosmetic composition according to claim 1, having a dynamic viscosity at 22° C. greater than 700 mPa·s.

15. (canceled)

16. (canceled)

17. A non-therapeutic cosmetic treatment method for skin and/or hair hygiene and/or care, by means of a cosmetic composition according to claim 1, comprising successive steps consisting of:
applying said composition on the skin and/or hair,
developing foam by rubbing the composition on the skin and/or the hair, and
rinsing.

18. The method as claimed in claim 17, wherein said skin is sensitive skin.
